(19) United States
(12) Patent Application Publication
(10) Pub. No.: US 2015/0080825 A1
(43) Pub. Date: Mar. 19, 2015

(54) HIGH DENSITY ABSORBENT CORES HAVING IMPROVED BLOOD WICKING

(71) Applicant: GP Cellulose GmbH, Zug (CH)

(72) Inventors: Darold Tippey, Atlanta, GA (US); Arthur J. Nonni, Peachtree City, GA (US)

(73) Assignee: GP Cellulose GmbH, Zug (CH)

(21) Appl. No.: 14/391,760

(22) PCT Filed: Apr. 10, 2013
(86) PCT No.: PCT/US13/35987

§ 371 Date: Oct. 10, 2014

Related U.S. Application Data
(60) Provisional application No. 61/686,730, filed on Apr. 11, 2012.
(60) Provisional application No. 61/794,738, filed on Mar. 15, 2013.

(51) Int. Cl. A61F 13/53; A61F 13/15; A61L 15/28

(52) U.S. Cl. CPC A61F 13/53; A61L 15/28; A61F 13/15; A61L 2400/04; A61F 2013/15422; A61F 2013/15463; A61F 2013/15365; A61F 2013/530007
USPC 604/375; 502/7

(57) ABSTRACT

The present disclosure describes absorbent cores and absorbent products that include highly compressed oxidized fibers that result in improved fluid handling, e.g. blood wicking properties, improved dimensional stability, improved rewet and better acquisition than comparable standard kraft pulp fiber devices.

This disclosure relates to the production of absorbent structures having improved blood wicking properties. This disclosure further relates to absorbent cores or bodies that are used in the production of absorbent devices or products. More particularly, the present disclosure describes absorbent cores or bodies that will come into contact with blood or blood products, e.g., wound care and feminine hygiene products. Contrary to what is seen with traditional fluff pulp, surprisingly, the absorbent structures of the invention exhibit improved fluid handling, e.g., blood wicking, as the density of the structure increases. Further, the absorbent structures of the present disclosure exhibit improved dimensional stability upon compression resulting in less rebound in a dry state and less growth upon fluid contact.

As used herein “absorbent structure” refers to any configuration of a fibrous absorbent structure that may come in contact with blood and blood products. Further as used herein “absorbent core” and “absorbent body,” are interchangeable and refer to fluff pulp that can be incorporated into an absorbent product. Absorbent cores and bodies are well understood in the art and are currently used in diapers, feminine hygiene products, adult incontinence products and the like.

The absorbent structures, as described herein, are made with fluff pulp comprising fiber produced as described in published International Application WO 2010/138941, which corresponds to U.S. patent application Ser. No. 13/322,419, both of which are incorporated by reference herein in their entirety. Absorbent structures, cores, and bodies as described herein may be made in their entirety from the fiber described in WO 2010/138941, or they may also include any art recognized fiber for use in absorbent structures. If other art recognized fibers are present, those fibers may be mixed together with fibers of International Application WO 2010/138941 to form a homogeneous body or they may be presented in one or more layers. If presented in layers, each layer may also include one or more fibers, mixed or layered.

Advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

FIG. 1 is a graph showing the average wetted area results for Examples 1 and 2.

FIG. 2 is a graph showing the average rewet results for Examples 1 and 2.

FIG. 3 is a graphical comparison of the samples of Examples 3 and 4, including the relevant data points.

FIG. 4 is a graphical comparison of the samples of Examples 3 and 4.

FIG. 5 is a graph of the average acquisition in seconds for the samples from Examples 3 and 4.

FIG. 6 is a graph of the average wetted area as a percent for the samples from Examples 3 and 4,

FIG. 7 is a graph of the calculated pad capacity in mls for the samples from Examples 3 and 4.

FIG. 8 is a graph of the average rewet in grams for the samples from Examples 3 and 4.

FIG. 9 is a summary graph of the average acquisition time in seconds for the samples from Examples 3 and 4.

FIG. 10 is a summary graph of the average wetted area as a percent for the samples from Examples 3 and 4.

FIG. 11 is a summary graph of the average pad capacity in mls for the samples from Examples 3 and 4.

FIG. 12 is a summary graph of the average rewet in grams for the samples from Examples 3 and 4,

FIG. 13 is a summary graph comparing the percent bulk thickness growth of an absorbent structure of the invention when insulted versus that of a standard fluff pulp structure when insulted, at five different product densities.

FIG. 14 is a summary graph comparing the thickness in mm of an absorbent structure of the invention when insulted versus a standard fluff pulp structure when insulted, at five different product densities.

### DESCRIPTION

Reference will now be made in detail to the present embodiments (exemplary embodiments) of the invention, examples of which are illustrated in the accompanying drawings.

Absorbent structures of this disclosure are fibrous structures that will contact blood and blood products. It is recognized that due to the viscosity and/or complex nature of blood it is difficult to effectively absorb blood using materials and structures that are quite successful at absorbing other fluids, such as urine. Thus, there is a need for cellulose fiber structures that can quickly and efficiently wick blood and hold that blood within the structure.

According to one embodiment of the present disclosure, the absorbent structures are absorbent cores or bodies for use in absorbent products having blood contact, including but not limited to feminine hygiene products and wound care items. Feminine hygiene products include but are not limited to sanitary napkins, and tampons. Heretofore, manufacturers of feminine hygiene products have been limited in their ability to produce thinner more compressed structures for blood absorption since compression of the fiber interferes with the uptake of the blood. Incontinence devices by contrast, are available as thinner products. These thinner incontinence products often use an acquisition layer to balance the need for quicker uptake against the wicking and holding properties of the compressed thinner structure. Acquisition layers have not been seen favorably when the fluid to be absorbed is blood.

According to another embodiment of the present disclosure, the absorbent structures can also be any structure of configuration where the absorption of blood and blood products is required. These structures may find use in, without limitation, wound care items including bandaids, bandages, pads, gauze, and any other dressings, as well as other medical fabrics including medical gowning, medical drapes and bed pads.

According to yet another embodiment of the disclosure, the absorbent structures of this disclosure can be used in environments where blood clean-up may be necessary, for example, in operating rooms, health clinics, dental offices, or accident scenes. The absorbent structures of the present invention, in addition to providing excellent blood uptake and absorption, have antimicrobial properties. Thus, the use of absorbent structures of the present invention in towels or absorbent pads used to clean up bodily fluid or blood will not only improve fluid handling, e.g., blood wicking and retention, in the absorbent structure, but will also reduce the growth of microbials on the structure after its use.

The structures and cores of this disclosure are formed from fibers that have been subjected to an oxidation treatment during bleaching, for example, the oxidation treatment may comprise a copper or iron catalyzed peroxide treatment in an acidic environment. These fibers, along with their characteristics, are described in U.S. patent application Ser. No. 13/322,419, which is incorporated herein by reference in its entirety. The oxidation of these fibers causes a change in the fibers chemical functionality. Specifically, the fibers have more aldehydic and carboxylic functionality than standard fluff pulp. Because of the changes to the chemical nature of the fibers, these fibers are compressible and have excellent odor control. The use of this fiber to make fluff pulp or an absorbent core was described in prior published application International Application WO 2010/138941. Unrecognized in that prior work, and quite surprising, is that when the absorbent fluff core is compressed to higher densities, it achieves superior results.

When these fibers are formed into a structure or body and are compressed, they have excellent dimensional stability, while remaining flexibility and exhibiting improved performance. Without wishing to be bound by theory, it is believed that the fiber used to produce the absorbent structure described herein is more three-dimensional than standard kraft fiber. By this, we mean that the fiber exhibits kink and curl not only in the x-y plane, but also in the z-plane. This increased three-dimensionality, coupled with dimensional stability upon compression, provides an absorbent body with better fluid handling characteristics. Absorbent bodies as described herein are characterized by better fluid uptake, i.e., the fluid or blood moves more quickly in a vertical direction through the core toward the bottom of the core and better fluid wicking, i.e., horizontal spread of the fluid toward the edges of the body. The wicked fluid of the absorbent bodies of this disclosure remains lower (further from the user side) within the body structure than is seen in bodies produced from standard fluff pulp. This fluid/blood profile results in faster fluid uptake, less rewet and larger capacity for the absorbent core.

The absorbent bodies as disclosed retain their dimensional stability after being insulted. More specifically, one reason the fluid moves toward the bottom of the core and wicks outward is because the absorbent bodies maintain strong dimensional stability, i.e., they don't swell or grow like standard fluff pulp. This structural stability causes the fluid to be forced outward to the edges of the device. This dimensional stability upon insult provides for a more comfortable product in use as the product remains as absorbent as a standard fluff body, but remains thinner making it more comfortable for the wearer.

The cores as described, when compared to cores made with fibers not subjected to an oxidation step, i.e., standard fluff pulp, can exhibit improved flexibility (especially when used in the bending side of a multilayer core), improved dimensional stability after insult, improved rewets with blood (especially when the disclosed fiber is placed in the top layer), improved wet and dry strength (again especially when the disclosed fiber is placed in the top layer), and better elongation.

The structures described may be produced in any art recognized manner, including a dry-forming technique, an air-forming technique, a wet-forming technique, a foam-forming technique, or the like, as well as combinations thereof. Methods and apparatus for carrying out such techniques are well known in the art. According to one embodiment, the core as described is produced by air-laying or air-forming the absorbent structure.

Absorbent cores or bodies as described can be compressed to a density of at least about 0.15 g/cm³, such as at least about 0.20 g/cm³, such as at least about 0.25 g/cm³. The structures may be compressed to at least about 0.35 g/cm³, such as about 0.45 g/cm³, such as about 0.5 g/cm³. The performance of the fibers at increased density allows the production of thinner core structure. The absorbent cores have good dimensional stability at these compression densities, making them subject to minimal rebound. Thinner structures, typically referred to as “ultra thin” products, provide better comfort and discreteness to the user.

The absorbent cores as described may be a single of multi-layered structures and may include fiber of the invention in one or more of a fluid acquisition layer, a distribution layer, a wicking layer and/or a storage layer. The absorbent cores as described perform best when they are produced solely from the oxidized fiber. However, for cost and other reasons, the skilled artisan may include other fiber in one or more of the product layers. Absorbent cores or bodies of the present invention may include one or more surface active agents to aide in processing or product characteristics, such as softness.

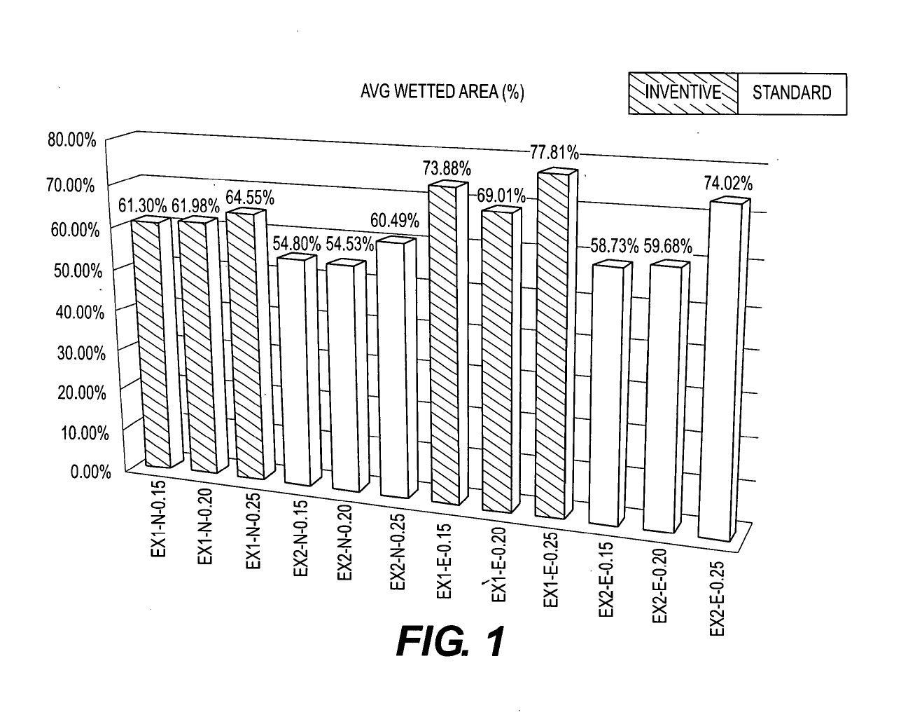

As can be seen in FIG. 1, the absorbent bodies of the present disclosure (Example 1) exhibit a higher average wetted area a body made with standard fluff pulp (Example 2). Further, FIG. 1 shows that the average wetted area increases as the density of the body gets higher.

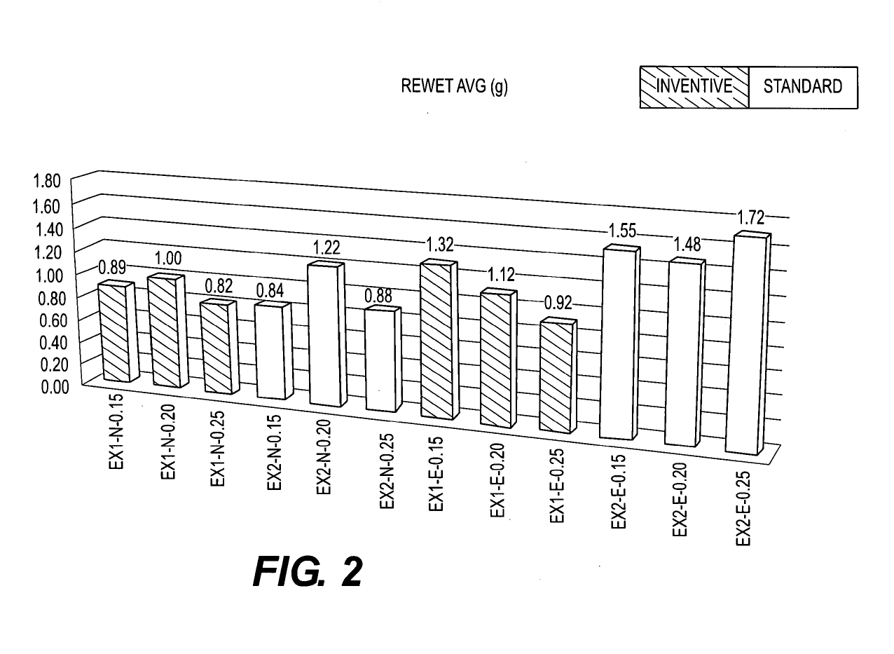

FIG. 2 shows that the average rewet for the bodies of FIG. 1 also improves as the density increases. As can be seen embossed cores (E) provide better rewet characteristics than unembossed cores (N).

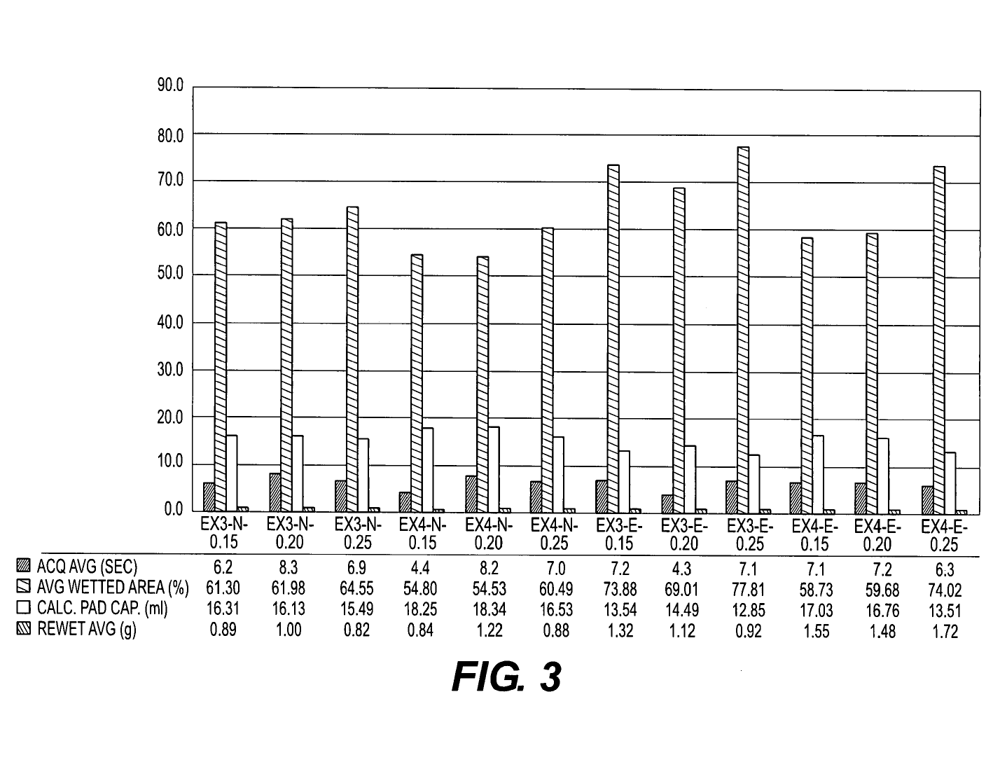

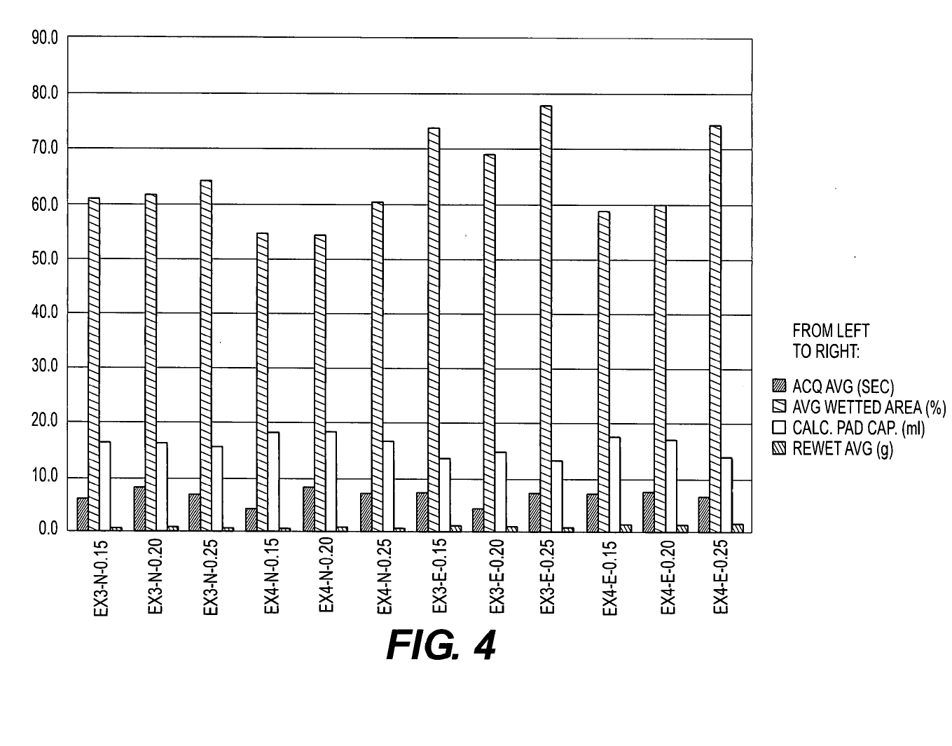

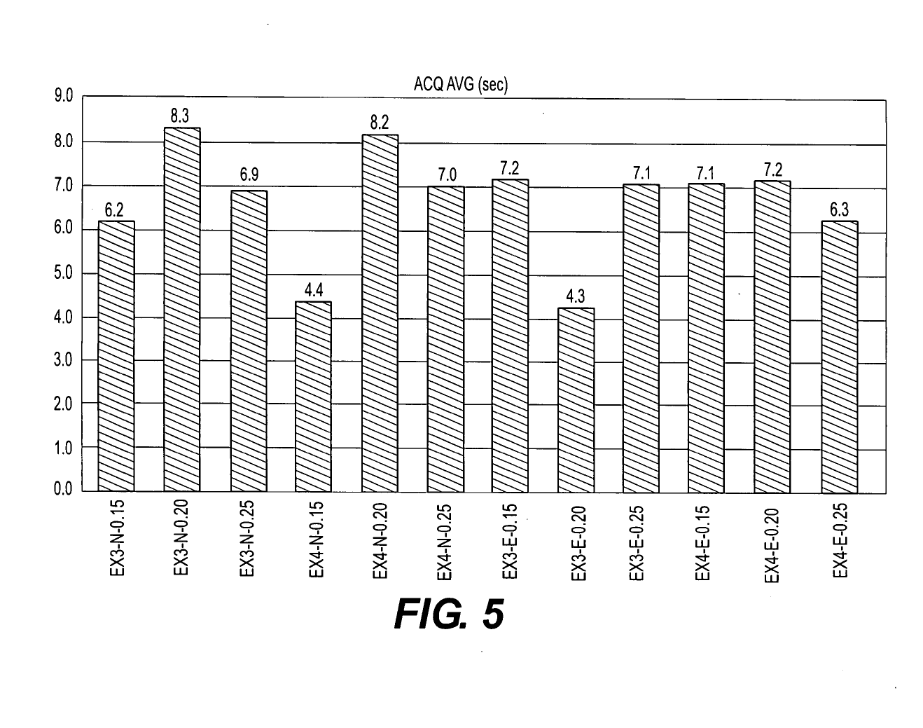

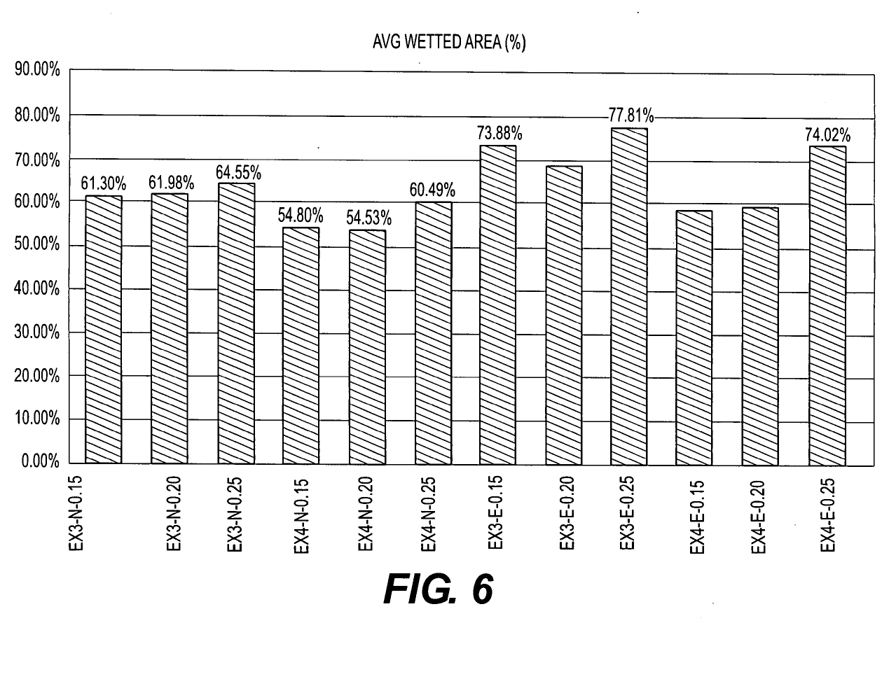

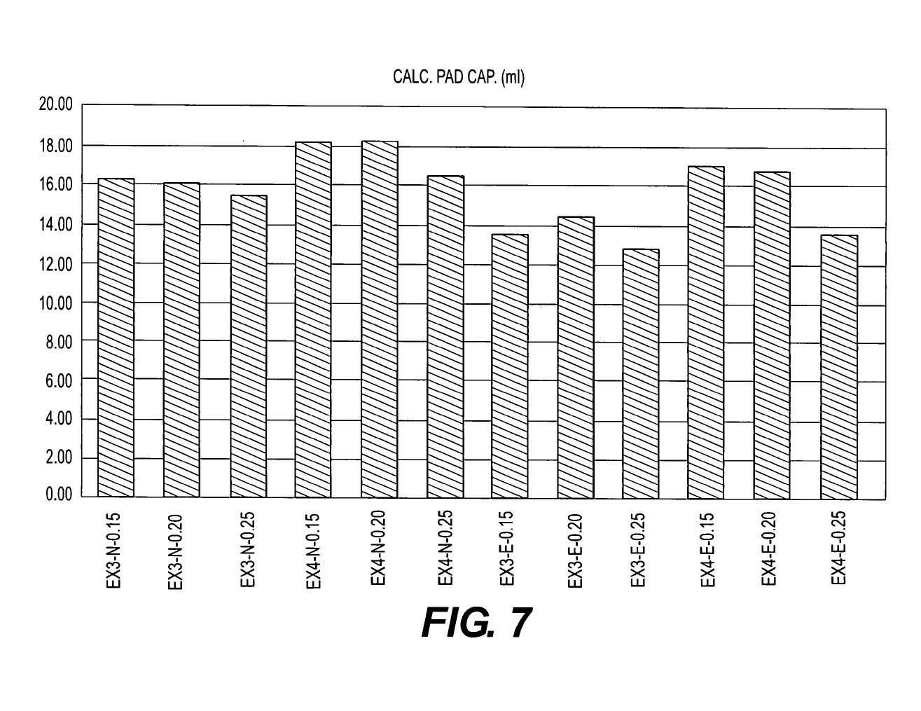

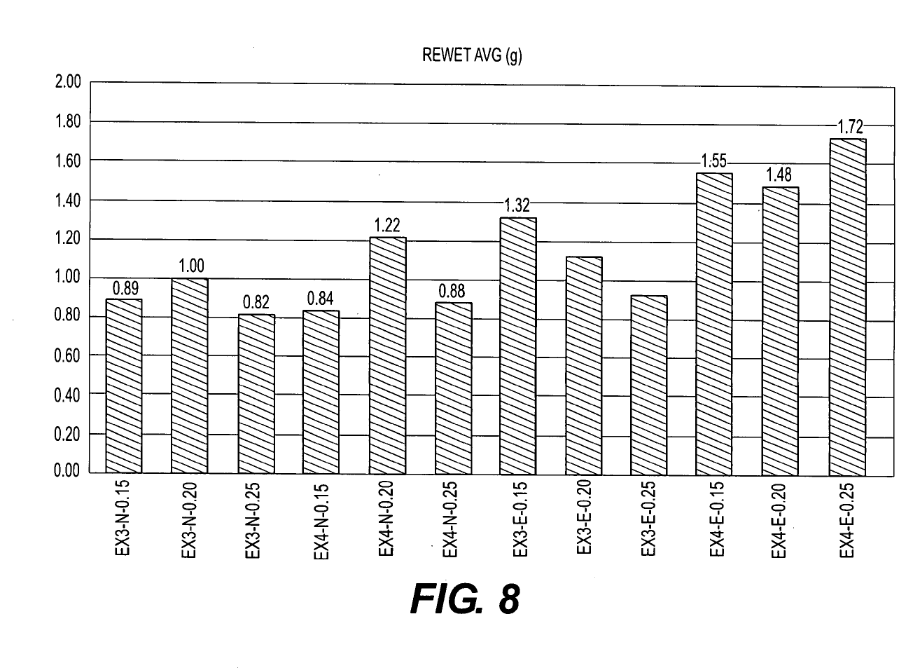

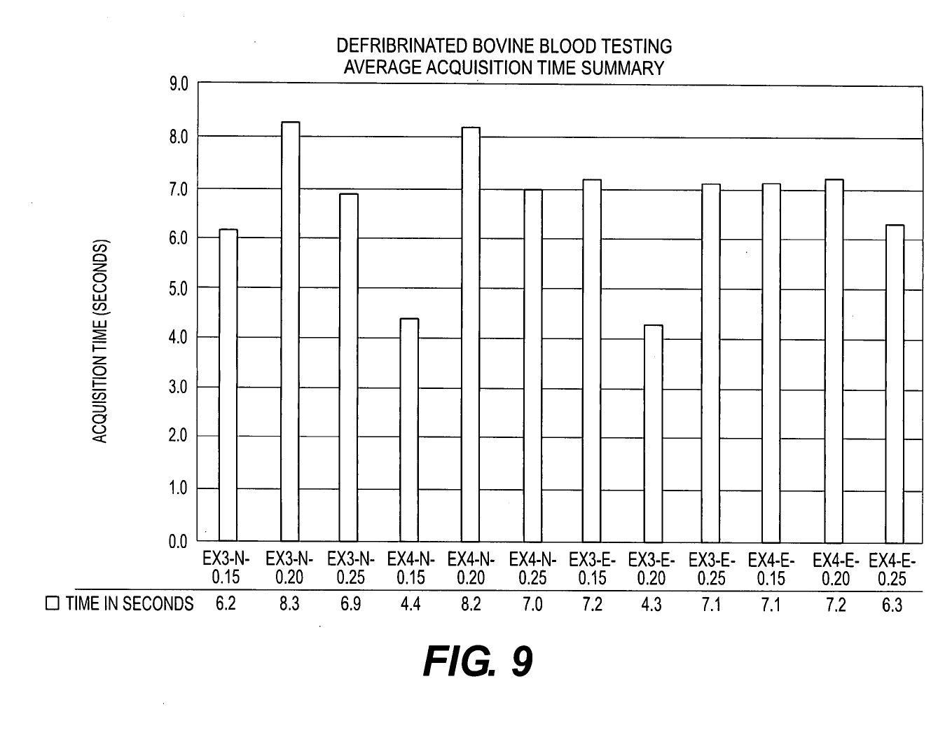

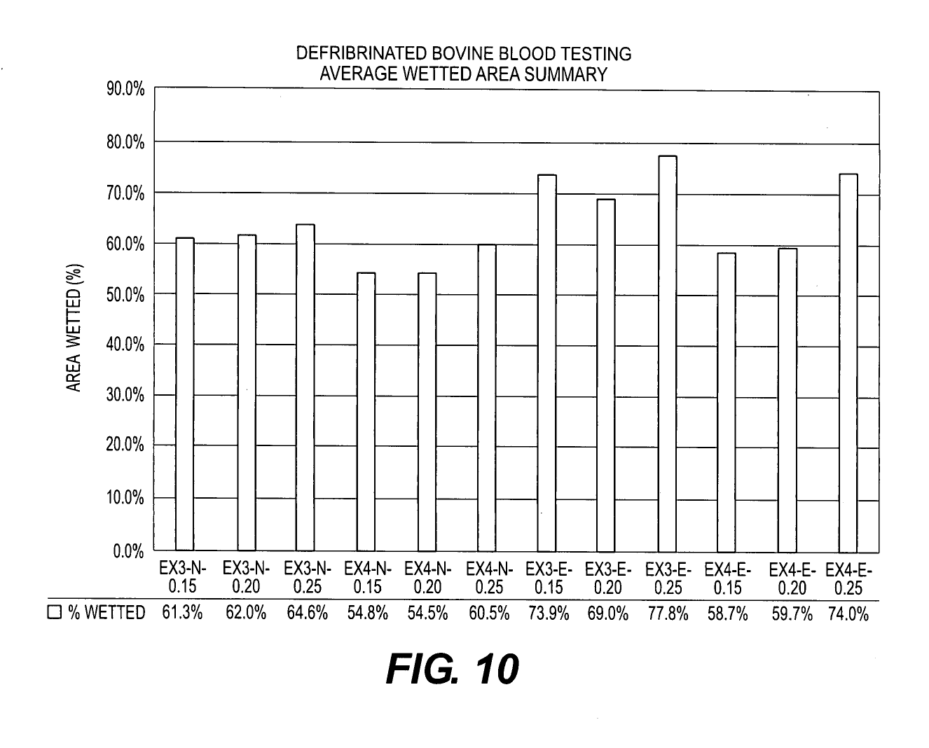

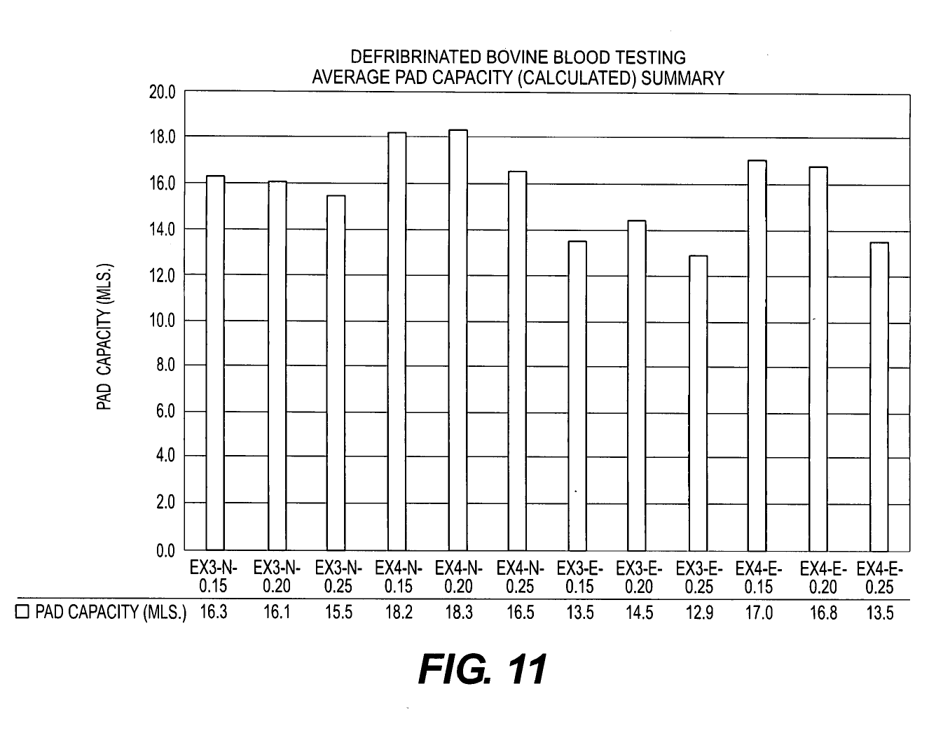

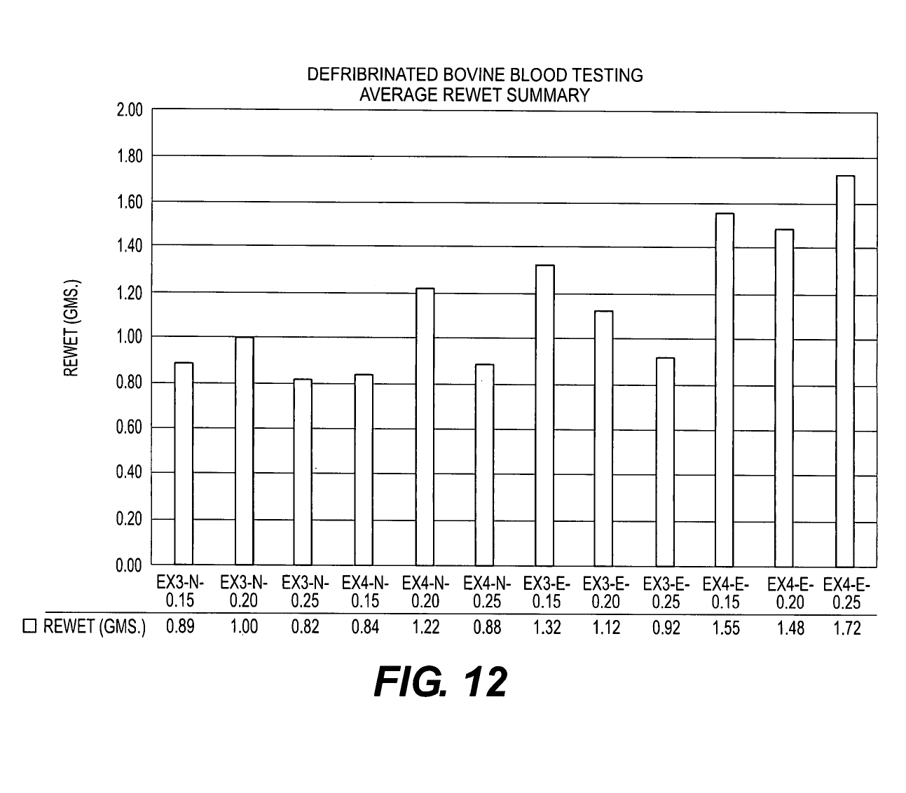

FIGS. 3 through 12 are directed to comparison examples 3 and 4. These figures again compare average wetted area and rewet of various bodies, but also provides an indication of acquisition average in seconds and the calculated pad capacity. As can be seen in FIGS. 3 through 12, the bodies of the instant invention (Example 3) show faster acquisition and better pad capacity than bodies made with standard fluff pulp (Example 4).

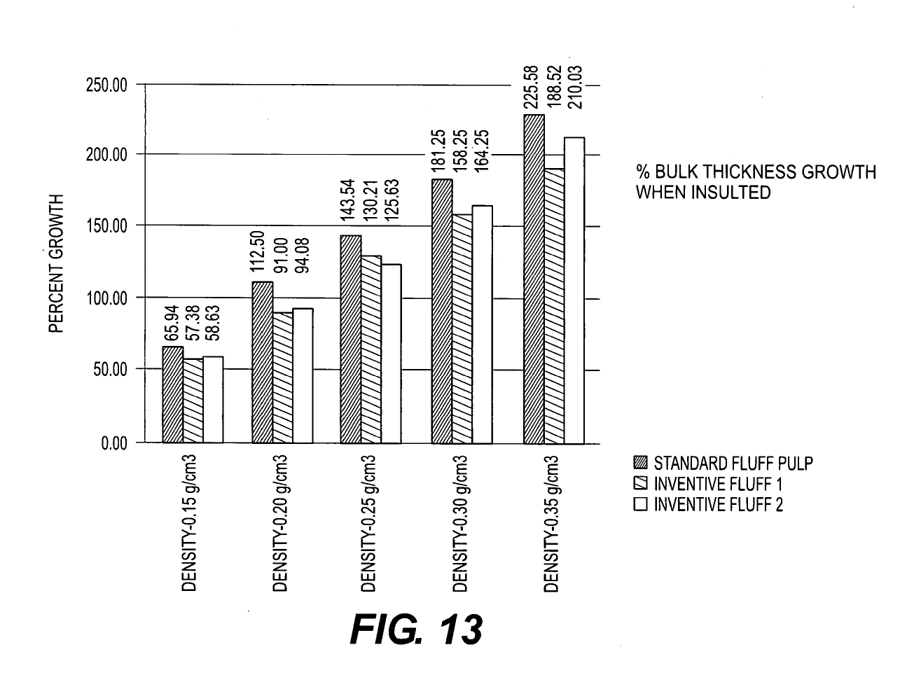

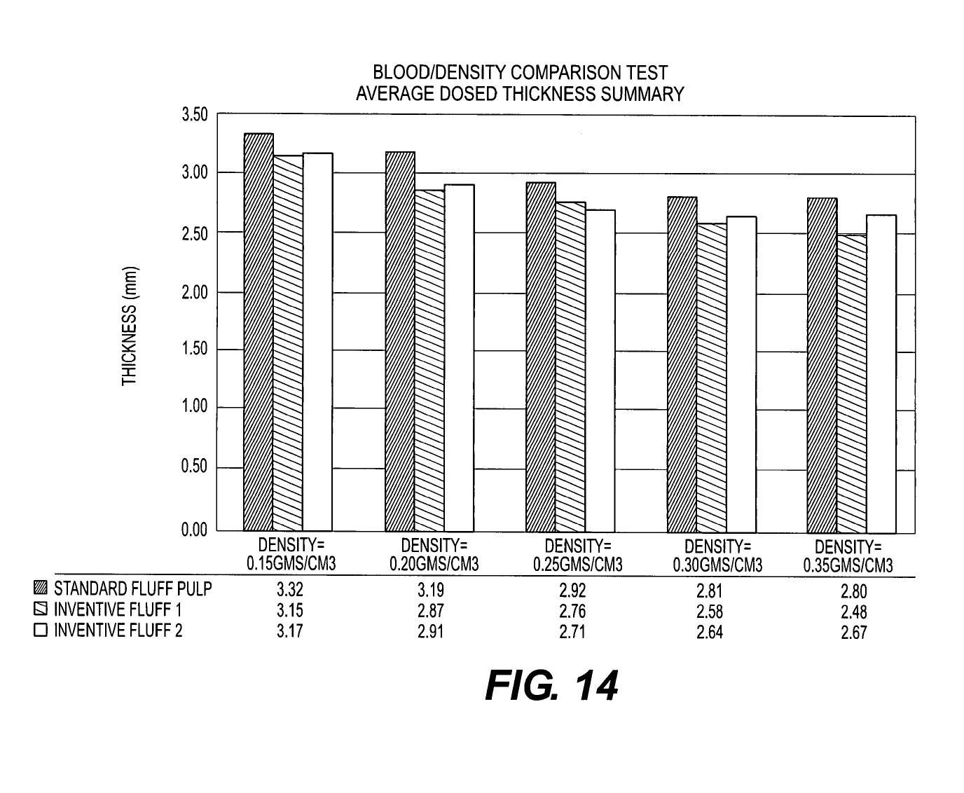

FIGS. 13 and 14 compare the bulk thickness growth of a body upon insult. As seen in FIG. 13, bodies were produced at five densities and then were insulted. In each example, the standard fluff pulp expanded more than the inventive body upon the application of liquid. As can be seen from FIG. 14, the bodies of the present disclosure were at least about 4% thinner upon insult than the standard fluff cores, for example, at least about 5% thinner, for example, at least about 6% thinner, for example at least about 8% thinner, for example at least about 10% thinner for example at least about 12% thinner for example at least about 15% thinner.

As can be seen from FIG. 13, the bodies according to the instant invention grew as much as about 20% less than standard fluff pulp, for example, as much as about 18% less, for example, as much as about 17% less, for example, as much as about 15% less, for example, as much as about 10% less, for example as much as about 8% less, for example as much as about 5% less than standard fluff pulp.

According to one embodiment, the absorbent structure of the instant invention is compressed to at least 0.15 g/m³ and is at least about 4% thinner upon insult than the standard fluff cores, for example, at least about 5% thinner, for example, at least about 6% thinner, for example at least about 8% thinner, for example at least about 10% thinner for example at least about 12% thinner for example at least about 15% thinner. According to another embodiment, the absorbent structure of the instant invention is compressed to at least about 0.15 g/m³ and grew as much as about 20% less than a structure of standard fluff pulp, for example, as much as about 18% less, for example, as much as about 17% less, for example, as much as about 15% less, for example, as much as about 10% less, for example as much as about 8% less, for example as much as about 5% less than standard fluff pulp.

According to another embodiment, the absorbent structure of the instant invention is compressed to at least about 0.20 g/m³ and is at least about 4% thinner upon insult than the standard fluff cores, for example, at least about 5% thinner, for example, at least about 6% thinner, for example at least about 8% thinner, for example at least about 10% thinner for example at least about 12% thinner for example at least about 15% thinner. According to another embodiment, the absorbent structure of the instant invention is compressed to at least about 0.20 g/m³ and grew as much as about 20% less than a structure of standard fluff pulp, for example, as much as about 18% less, for example, as much as about 17% less, for example, as much as about 15% less, for example, as much as about 10% less, for example as much as about 8% less, for example as much as about 5% less than standard fluff pulp.

According to another embodiment, the absorbent structure of the instant invention is compressed to at least about 0.25 g/m³ and is at least about 4% thinner upon insult than the standard fluff cores, for example, at least about 5% thinner, for example, at least about 6% thinner, for example at least about 8% thinner, for example at least about 10% thinner for example at least about 12% thinner for example at least about 15% thinner. According to another embodiment, the absorbent structure of the instant invention is compressed to at least about 0.25 g/m³ and grew as much as about 20% less than a structure of standard fluff pulp, for example, as much as about 18% less, for example, as much as about 17% less, for example, as much as about 15% less, for example, as much as about 10% less, for example as much as about 8% less, for example as much as about 5% less than standard fluff pulp.

According to another embodiment, the absorbent structure of the instant invention is compressed to at least about 0.30 g/m³ and is at least about 4% thinner upon insult than the standard fluff cores, for example, at least about 5% thinner, for example, at least about 6% thinner, for example at least about 8% thinner, for example at least about 10% thinner for example at least about 12% thinner for example at least about 15% thinner. According to another embodiment, the absorbent structure of the instant invention is compressed to at least about 0.30 g/m³ and grew as much as about 20% less than a structure of standard fluff pulp, for example, as much as about 18% less, for example, as much as about 17% less, for example, as much as about 15% less, for example, as much as about 10% less, for example as much as about 8% less, for example as much as about 5% less than standard fluff pulp.

According to another embodiment, the absorbent structure of the instant invention is compressed to at least about 0.35 g/m³ and is at least about 4% thinner upon insult than the standard fluff cores, for example, at least about 5% thinner, for example, at least about 6% thinner, for example at least about 8% thinner, for example at least about 10% thinner for example at least about 12% thinner for example at least about 15% thinner. According to another embodiment, the absorbent structure of the instant invention is compressed to at least about 0.35 g/m³ and grew as much as about 20% less than a structure of standard fluff pulp, for example, as much as about 18% less, for example, as much as about 17% less, for example, as much as about 15% less, for example, as much as about 10% less, for example as much as about 8% less, for example as much as about 5% less than standard fluff pulp.

As used herein standard cellulose pulp refers to fluff pulp that does not include oxidized fibers. When comparing results against standard fluff pulp as used in this application, one would compare the device or body of interest against a device or body having the same configuration as the device or body of interest, but the comparative device or body would use only commercially available kraft pulp.

The fiber may, in some embodiments, be treated with a surface active agent. The surface active agent for use in the present invention may be solid or liquid. The surface active agent can be any surface active agent, including by not limited to softeners, debonders, and surfactants that is not substantive to the fiber, i.e., which does not interfere with its specific absorption rate. As used herein a surface active agent that is “not substantive” to the fiber exhibits an increase in specific absorption rate of 30% or less as measured using the pfi test as described herein. According to one embodiment, the specific absorption rate is increased by about 25% or less, such as about 20% or less, such as about 15% or less, such as about 10% or less. Not wishing to be bound by theory, the addition of surfactant causes competition for the same sites on the cellulose as the test fluid. Thus, when a surfactant is too substantive, it reacts at too many sites reducing the absorption capability of the fiber.

As used herein PFI is measured according to SCAN-C-33:80 Test Standard, Scandinavian Pulp, Paper and Board Testing Committee. The method is generally as follows. First, the sample is prepared using a PFI Pad Former. Turn on the vacuum and feed approximately 3.01 g fluff pulp into the pad former inlet. Turn off the vacuum, remove the test piece and place it on a balance to check the pad mass. Adjust the fluff mass to 3.00±0.01 g and record as Mass_{dry}. Place the fluff into the test cylinder. Place the fluff containing cylinder in the shallow perforated dish of an Absorption Tester and turn the water valve on. Gently apply a 500 g load to the fluff pad while lifting the test piece cylinder and promptly press the start button. The Tester will fun for 30 s before the display will read 00.00. When the display reads 20 seconds, record the dry pad height to the nearest 0.5 mm (Height_{dry}). When the display again reads 00.00, press the start button again to prompt the tray to automatically raise the water and then record the time display (absorption time, T). The Tester will continue to run for 30 seconds. The water tray will automatically lower and the time will run for another 30 S. When the display reads 20 s, record the wet pad height to the nearest 0.5 mm (Height_{wet}). Remove the sample holder, transfer the wet pad to the balance for measurement of Mass_{wet} and shut off the water valve. Specific Absorption Rate (s/g) is T/Mass_{dry}. Specific Capacity (g/g) is (Mass_{wet}−Mass_{dry})/Mass_{dry}. Wet Bulk (cc/g) is [19.64 cm²×Height_{wet}/3]/10. Dry Bulk is [19.64 cm²×Height_{dry}/3]/10. The reference standard for comparison with the surfactant treated fiber is an identical fiber without the addition of surfactant.

It is generally recognized that softeners and debonders are often available commercially only as complex mixtures rather than as single compounds. While the following discussion will focus on the predominant species, it should be understood that commercially available mixtures would generally be used in practice. Suitable softener, debonder and surfactants will be readily apparent to the skilled artisan and are widely reported in the literature.

Suitable surfactants include cationic surfactants, anionic, and nonionic surfactants that are not substantive to the fiber. According to one embodiment, the surfactant is a non-ionic surfactant. According to one embodiment, the surfactant is a cationic surfactant. According to one embodiment, the surfactant is a vegetable based surfactant, such as a vegetable based fatty acid, such as a vegetable based fatty acid quaternary ammonium salt. Such compounds include DB999 and DB1009, both available from Cellulose Solutions. Other surfactants may be including, but not limited to Berol 388 an ethoxylated nonylphenol ether from Akzo Nobel.

Biodegradable softeners can be utilized. Representative biodegradable cationic softeners/debonders are disclosed in U.S. Pat. Nos. 5,312,522; 5,415,737; 5,262,007; 5,264,082; and 5,223,096, all of which are incorporated herein by reference in their entirety. The compounds are biodegradable diesters of quaternary ammonia compounds, quaternized amine-esters, and biodegradable vegetable oil based esters functional with quaternary ammonium chloride and diester dierucyldimethyl ammonium chloride and are representative biodegradable softeners.

The surfactant is added in an amount of up to about 6 lbs/ton, such as from about 0.5 lbs/ton to about 3 lbs/ton, such as from about 0.5 lbs/ton to about 2.5 lbs/ton such as from about 0.5 lbs/ton to about 2 lbs/ton, such as less than about 2 lbs/ton.

The surface active agent may be added at any point prior to forming rolls, bales, or sheets of pulp. According to one embodiment, the surface active agent is added just prior to the headbox of the pulp machine, specifically at the inlet of the primary cleaner feed pump.

According to one embodiment, the oxidized fiber is formed into a core structure which is compressed in a nip. The compressed core can be included in any absorbent product, for example, a feminine hygiene product, wound dressings, bed pads or any other product that would come into contact with blood. The absorbent cores and bodies of the instant disclosure can be measure for formation index using an M/K Formation tester and following the manufacturer's procedure. Absorbent bodies and cores of the instant disclosure generally show a 15% to 20% improvement in formation index over cores made with standard kraft fiber, for example, at least about a 10% improvement, for example, at least about a 15% improvement, for example, at least about a 17% improvement, for example, at least about a 20% improvement.

According to one embodiment, fiber is air-laid to form an absorbent structure. According to another embodiment, the air-laid fiber is changed between the front (user side) to the back. Oxidized fiber used in the back layer provides good dimensional stability, improved flexibility and good fluid retention. Oxidized fiber in the central layer of the core provides improved fluid uptake, wicking and rewet. Oxidized fiber in the top layer provides improved fluid uptake. Changes to the fiber are not always compositional. In one embodiment, the fiber of the top layer is treated with a surface active agent, while the center and back layers are not. According to another embodiment, the top and back layers are treated with a surface active agent, while the middle layer is not. The multi-layer core may also be compressed in a nip.

In one embodiment, the compressed structure of the invention may be used as an acquisition layer in an absorbent product. In another embodiment, the structure may be used as a retention layer at the back of the absorbent core. In another embodiment, the structure may be selectively compressed to provide an integral acquisition layer or areas of acquisition within a core or body structure.

The structures and cores of the present invention may be unembossed or may be embossed with any art recognized pattern. Appropriate patterns may include micro embossments, macro embossments and/or signature embossments. Signature embossments refer to emboss patterns or elements that designate source or origin.

In some embodiments, the cores as described may include the described fluff pulp in combination with other materials that are generally found in absorbent cores. For example, the absorbent core may include other natural fibers, synthetic fibers, woven or nonwoven sheets, scrim netting or other stabilizing structures, superabsorbent material, binder materials, surfactants, selected hydrophobic materials, pigments, lotions, odor control agents or the like, as ell as combinations thereof.

If SAP is combined with the fluff pulp to create an absorbent core, as described, any art recognized material may be used. SAP may be chosen from natural, synthetic, and modified natural polymers and materials. SAP can be inorganic materials, such as silica gels, or organic compounds, such as crosslinked polymers. Typically, a superabsorbent material is capable of absorbing at least about 10 times its weight in liquid, and preferably is capable of absorbing more than about 25 times its weight in liquid. Suitable SAP includes for example, Hysorb™ sold by company BASF, Aqua Keep® sold by the company Sumitomo, and FAVOR®, sold by the company Evonik. SAP is held well in these absorbent structures. SAP is retained by the interlocking and packing of the oxidized fibers.

Absorbent products made using the absorbent cores as described herein will often include the core between a barrier/backsheet material (often a film) and a body-side liner (a nonwoven material).

### EXAMPLES

### Example 1

Fiber produced according to WO 2010/138941 was converted into fluff pulp of 300 g/m². The fluff was cut into cores of 6×16 cms. The cores were compressed to 0.15 g/cm³, 0.20 g/m³ and 0.25 g/cm³, respectively. The cores were placed on a poly film and insulted with 10 mls of defibrinated bovine blood over a 10 second period. The intake rate (sec), average percent wetted area of the sample and rewet transfer to filter paper (g) were measured. The average percent wetted area of the sample was measured after 10 minutes. Three embossed and three unembossed sample were tested.

### Example 2

### Comparative

Samples of standard fiber were used to produce fluff pulp of 300 g/m². As in Example 1, the cores were cut, compressed and placed on a poly film and insulted with bovine blood. Again, the intake rate (sec), average percent wetted area of the sample and rewet transfer to filter paper (g) were measured. Three embossed and three unembossed sample were tested

The results for Examples 1 and 2 can be seen in the graphs of FIGS. 1 and 2. As can be seen in FIGS. 1 and 2, the cores of the present invention had better distributed blood when compared to the standard fluff pulp of Example 2. The comparative samples had a percent average blood stained area of only 54.53%, while the samples of example 1 had a percent average blood stained area of 61.98%. Also, the inventive cores transferred less blood to the filter paper and quite surprisingly, the inventive cores performed better as the density was increased.

For the inventive cores the blood quickly wicked through the pulp to the bottom side of the core. The comparative samples had a percent average blood stained area of only 60.49%, while the samples of Example 1 had a percent average blood stained area of 64.55%. FIG. 2 shows that each of the inventive samples, with a single exception, had lower rewet than samples made from standard fluff pulp. Of particular note is that Example 1 at a density of 0.25 g/m³, the rewet was significantly lower than that of its comparative Sample 2 at 0.25 g/m³, i.e., 1.72 grams of released fluid for the standard sample, but only 0.92 grams of released fluid for the inventive sample.

As used in all of the Examples, a sample noted with an “N” has not been embossed, while a sample with an “E” has been embossed.

### Example 3

Fiber produced according to WO 2010/138941 was converted into fluff pulp of 300 g/m² and used to generate an unbonded airlaid web deposited on 18 g/m² tissue. Rolls at 0.15, 0.20 and 0.25 g/cm³ were produced. Rolls that were embossed were subsequently recompressed to achieve the correct densities.

Simulated feminine pads were made from fabrics sampled from the rolls and adjusted to the correct density. These “feminine pads” included a diaper-type film and a bicomponent fiber coverstock. A 10 ml aliquot of bovine blood was applied to the center of the pad over a 10 second interval and the time taken to penetrate fully into the pad was recorded. After 10 minutes, the samples were scanned, looking at the bottom side, to record the total area stained by the blood. These data were used to calculate a theoretical total pad capacity. The samples were then covered with blotter paper and loaded together with appropriated weight to obtain a rewet average for all of the samples together.

Results can be seen in FIGS. 3-12.

### Example 4

### Comparative

Standard fiber was converted into fluff pulp of 300 g/m² and used to generate an unbonded airlaid web deposited on 18 g/m² tissue. Rolls at 0.15, 0.20 and 0.25 g/cm³ densities were produced. Rolls that were embossed were subsequently recompressed to achieve the correct densities.

Simulated feminine pads were made from fabrics sampled from the rolls and adjusted to the correct density. These included a diaper-type film and a bicomponent fiber coverstock. A 10 ml aliquot of bovine blood was applied to the center of the pad over a 10 second interval and the time taken to penetrate fully into the pad was recorded. After 10 minutes, the samples were scanned, looking at the bottom side, to record the total area stained by the blood. These data were used to calculate a theoretical total pad capacity. The samples were then covered with blotter paper and loaded together with appropriated weight to obtain a rewet average for all of the samples together.

Results can be seen in FIGS. 3-12.

### Example 5

### Defibrinated Bovine Blood Testing of Multilayer Absorbent Sheets

Five different airlaid multilayer sheets were prepared and cut into 200 4×8 inch rectangles. The differing sets were labeled as shown in Table 1. Where noted, conventional sheets were treated with TQ-2021 and modified sheets were treated with TQ-2028, both surface active agents supplied by Ashland, Inc.

TABLE 1

| | | Middle | |
|---|---|---|---|
| Sheet | Top Layer | Layer | Bottom Layer |
| Control 1 | Conventional | Non GP | Conventional with TQ- |
| | with TQ-2021 | untreated | 2021 |
| Trial 1 | Conventional | Non GP | Modified with TQ-2028 |
| | with TQ-2021 | untreated | |
| Trial 2 | Conventional | Modified | Modified with TQ-2028 |
| | with TQ-2021 | | |
| Trial 3 | Modified with | Modified | Conventional with TQ- |
| | TQ-2028 | | 2021 |
| Trial 4 | Modified with | Non GP | Conventional with TQ- |
| | TQ-2028 | untreated | 2021 |

The sheets were profiled. The results are shown in Table 2.

TABLE 2

| | | | Front | Back | |
|---|---|---|---|---|---|
| Product Profile | | Front | Middle | Middle | Back |
| Control 1 | Basis Wt. (g/m2) | 173 | 171 | 174 | 168 |
| | Density (g/cc) | 0.20 | 0.19 | 0.20 | 0.19 |
| | Caliper (cm) | 0.09 | 0.09 | 0.09 | 0.09 |
| Trial 1 | Basis Wt. (g/m2) | 175 | 171 | 172 | 174 |
| | Density (g/cc) | 0.20 | 0.19 | 0.19 | 0.20 |
| | Caliper (cm) | 0.09 | 0.09 | 0.09 | 0.09 |
| Trial 2 | Basis Wt. (g/m2) | 168 | 171 | 170 | 172 |
| | Density (g/cc) | 0.20 | 0.19 | 0.19 | 0.20 |
| | Caliper (cm) | 0.09 | 0.09 | 0.09 | 0.09 |
| Trial 3 | Basis Wt. (g/m2) | 174 | 173 | 173 | 167 |
| | Density (g/cc) | 0.20 | 0.20 | 0.20 | 0.19 |
| | Caliper (cm) | 0.09 | 0.09 | 0.09 | 0.09 |
| Trial 4 | Basis Wt. (g/m2) | 181 | 182 | 177 | 177 |
| | Density (g/cc) | 0.20 | 0.20 | 0.20 | 0.20 |
| | Caliper (cm) | 0.09 | 0.09 | 0.09 | 0.09 |

Tests were conducted by Materials Testing Service of Kalamazoo, Mich., using their own test equipment and procedures for acquisition rate, capacity, and rewet properties using defibrinated bovine blood. The results are shown in Tables 3-5.

TABLE 3
Defibrinated Bovine Blood Testing Acquisition Time/Rate

| | Dose/pad @ | Acq Avg of | Acq Avg of | Acquisition |
|---|---|---|---|---|
| Product | 0.5 ml/sec | 5 (sec) | 30 (sec) | Rate (mls/sec) |
| Control 1 | 5.0 | 21.9 | 25.0 | 0.20 |
| Control 1 | 5.0 | 23.5 | | |
| Control 1 | 5.0 | 25.5 | | |
| Control 1 | 5.0 | 23.9 | | |
| Control 1 | 5.0 | 26.8 | | |
| Control 1 | 5.0 | 28.4 | | |
| Trial 1 | 5.0 | 24.4 | 26.6 | 0.19 |
| Trial 1 | 5.0 | 26.9 | | |
| Trial 1 | 5.0 | 27.1 | | |
| Trial 1 | 5.0 | 27.7 | | |
| Trial 1 | 5.0 | 26.8 | | |
| Trial 1 | 5.0 | 26.6 | | |
| Trial 2 | 5.0 | 21.9 | 18.2 | 0.27 |
| Trial 2 | 5.0 | 15.1 | | |
| Trial 2 | 5.0 | 14.9 | | |
| Trial 2 | 5.0 | 17.7 | | |
| Trial 2 | 5.0 | 19.0 | | |
| Trial 2 | 5.0 | 20.8 | | |
| Trial 3 | 5.0 | 25.6 | 26.0 | 0.19 |
| Trial 3 | 5.0 | 24.6 | | |
| Trial 3 | 5.0 | 25.1 | | |
| Trial 3 | 5.0 | 25.4 | | |
| Trial 3 | 5.0 | 27.1 | | |
| Trial 3 | 5.0 | 28.2 | | |
| Trial 4 | 5.0 | 26.5 | 27.6 | 0.18 |
| Trial 4 | 5.0 | 27.5 | | |
| Trial 4 | 5.0 | 28.7 | | |
| Trial 4 | 5.0 | 27.1 | | |
| Trial 4 | 5.0 | 28.6 | | |
| Trial 4 | 5.0 | 27.4 | | |

TABLE 4
Defibrinated Bovine Blood Testing Thickness (Dry/Wet)

| | Dry Thk | Dosed | Dosed |
|---|---|---|---|
| | Avg/Roll | Thk Avg | Thickness |
| Product | (mm) | of 30 (mm) | Change (%) |
| Control 1 | 0.80 | 1.04 | 29.2 |
| Trial 1 | 0.76 | 0.96 | 26.8 |
| Trial 2 | 0.75 | 0.94 | 26.5 |
| Trial 3 | 0.76 | 0.95 | 25.4 |
| Trial 4 | 0.78 | 0.98 | 25.8 |

TABLE 5
Defibrinated Bovine Blood Testing Rewet/Wetted Area/Capacity

| | | | Estimated |
|---|---|---|---|
| | Rewet | Wetted Area | Capacity |
| | Avg. Rewet/Pad | Avg Wetted | Calc. Avg Pad |
| Product | (g) | Area of 30 (%) | Cap. of 30 (ml) |
| Control 1 | 0.37 | 63.6 | 7.86 |
| Trial 1 | 0.38 | 64.0 | 7.81 |
| Trial 2 | 0.38 | 64.3 | 7.77 |
| Trial 3 | 0.32 | 65.7 | 7.61 |
| Trial 4 | 0.33 | 65.6 | 7.62 |

### Example 6

### Defibrinated Bovine Blood Testing of Individual Sheets

The defibrinated bovine blood acquisition rate, capacity, and rewet properties of sheets of various densities (0.15, 0.25, and 0.35 g/cm³) and basis weights (60, 150, 300 gsm) made from pulp produced from modified cellulose according to the disclosure and 10% bicomponent fiber was compared with sheets made from conventional kraft pulp. Tests were conducted by Materials Testing Service of Kalamazoo, Mich., using their own test equipment and procedures. The results are depicted in Tables 6-7 below.

TABLE 6
Defibrinated Bovine Blood Testing Acquisition Time and Thickness

| | | | | | |
|---|---|---|---|---|---|
| | Acq | | | Dosed | Dosed |
| | Avg | ACQUISITION | | Thk | Thk |
| Dose/pad | of 10 | RATE | Thickness | Avg | Change |
| @1 ml/sec | (sec) | (MLS/SEC.) | (mm) | (mm) | (%) |

Conventional

| | | | | | | |
|---|---|---|---|---|---|---|
| 60GSM - 0.15 | 2.0 | 8.6 | 0.23 | 0.40 | 0.57 | 42.50 |
| DENSITY | | | | | | |
| 60GSM - 0.25 | 2.0 | 9.9 | 0.20 | 0.24 | 0.62 | 158.33 |
| DENSITY | | | | | | |
| 60GSM - 0.35 | 2.0 | 14.1 | 0.14 | 0.17 | 0.52 | 205.88 |
| DENSITY | | | | | | |
| 150GSM - 0.15 | 5.0 | 7.3 | 0.69 | 1.00 | 1.74 | 74.00 |
| DENSITY | | | | | | |
| 150GSM - 0.25 | 5.0 | 12.1 | 0.41 | 0.60 | 1.23 | 105.00 |
| DENSITY | | | | | | |
| 150GSM - 0.35 | 5.0 | 34.8 | 0.14 | 0.43 | 0.96 | 123.26 |
| DENSITY | | | | | | |
| 300GSM - 0.15 | 10.0 | 5.0 | 2.02 | 1.95 | 3.08 | 57.95 |
| DENSITY | | | | | | |
| 300GSM - 0.25 | 10.0 | 19.2 | 0.52 | 1.20 | 2.22 | 85.00 |
| DENSITY | | | | | | |
| 300GSM - 0.35 | 10.0 | 58.0 | 0.17 | 0.87 | 1.53 | 75.86 |
| DENSITY | | | | | | |

Modified

| | | | | | | |
|---|---|---|---|---|---|---|
| 60GSM - 0.15 | 2.0 | 7.7 | 0.26 | 0.40 | 0.66 | 65.00 |
| DENSITY | | | | | | |
| 60GSM - 0.25 | 2.0 | 11.4 | 0.18 | 0.24 | 0.56 | 133.33 |
| DENSITY | | | | | | |
| 60GSM - 0.35 | 2.0 | 13.6 | 0.15 | 0.17 | 0.46 | 170.59 |
| DENSITY | | | | | | |
| 150GSM - 0.15 | 5.0 | 6.6 | 0.76 | 1.00 | 1.73 | 73.00 |
| DENSITY | | | | | | |
| 150GSM - 0.25 | 5.0 | 10.4 | 0.48 | 0.60 | 1.46 | 143.33 |
| DENSITY | | | | | | |
| 150GSM - 0.35 | 5.0 | 21.8 | 0.23 | 0.43 | 1.05 | 144.19 |
| DENSITY | | | | | | |
| 300GSM - 0.15 | 10.0 | 9.1 | 1.10 | 1.95 | 2.82 | 44.62 |
| DENSITY | | | | | | |
| 300GSM - 0.25 | 10.0 | 11.9 | 0.84 | 1.20 | 2.33 | 94.17 |
| DENSITY | | | | | | |
| 300GSM - 0.35 | 10.0 | 56.3 | 0.18 | 0.87 | 1.39 | 59.77 |
| DENSITY | | | | | | |

TABLE 7
Defibrinated Bovine Blood Testing Rewet/Wetted Area/Capacity

| | Rewet | Wetted Area | Estimated Capacity |
|---|---|---|---|
| | Rewet/Pad | Avg Wetted Area of | Calc. Avg Pad Cap. |
| Product | (g) | 10 (%) | of 10 (ml) |

Conventional

| | | | |
|---|---|---|---|
| 60GSM - 0.15 | 0.44 | 38.17 | 5.24 |
| DENSITY | | | |
| 60GSM - 0.25 | 0.48 | 40.44 | 4.95 |
| DENSITY | | | |
| 60GSM - 0.35 | 0.33 | 47.24 | 4.23 |
| DENSITY | | | |
| 150GSM - 0.15 | 0.73 | 34.14 | 14.64 |
| DENSITY | | | |
| 150GSM - 0.25 | 0.85 | 50.48 | 9.90 |
| DENSITY | | | |
| 150GSM - 0.35 | 0.67 | 64.63 | 7.74 |
| DENSITY | | | |
| 300GSM - 0.15 | 1.30 | 42.52 | 23.52 |
| DENSITY | | | |
| 300GSM - 0.25 | 1.20 | 55.08 | 18.16 |
| DENSITY | | | |
| 300GSM - 0.35 | 1.10 | 87.53 | 11.42 |
| DENSITY | | | |

Modified

| | | | |
|---|---|---|---|
| 60GSM - 0.15 | 0.32 | 38.02 | 5.26 |
| DENSITY | | | |
| 60GSM - 0.25 | 0.37 | 42.09 | 4.75 |
| DENSITY | | | |
| 60GSM - 0.35 | 0.27 | 49.78 | 4.02 |
| DENSITY | | | |
| 150GSM - 0.15 | 0.49 | 33.00 | 15.15 |
| DENSITY | | | |
| 150GSM - 0.25 | 0.76 | 49.02 | 10.20 |
| DENSITY | | | |
| 150GSM - 0.35 | 0.74 | 59.52 | 8.40 |
| DENSITY | | | |
| 300GSM - 0.15 | 1.07 | 40.05 | 24.97 |
| DENSITY | | | |
| 300GSM - 0.25 | 1.18 | 54.72 | 18.28 |
| DENSITY | | | |
| 300GSM - 0.35 | 0.81 | 99.44 | 10.06 |
| DENSITY | | | |

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-22. (canceled)

23. A method for absorbing blood comprising:
providing a compressed absorbent structure comprising fluff pulp comprising cellulosic fiber that has been oxidized with a copper or iron catalyst and a peroxide in an acidic environment within a bleaching sequence, and having a bulk thickness and a density of at least about 0.15 g/cm³; and
contacting the compressed absorbent structure with blood;
wherein the bulk thickness of the compressed absorbent structure grows by less than about 59% when contacted with 10 mls of blood over a 10 second period.

24. The method of claim 23, wherein the compressed absorbent structure has a density of at least about 0.20 g/cm³ and wherein the bulk thickness of the compressed absorbent structure grows by less than about 95% after contact with 10 mls of blood over a 10 second period.

25. The method of claim 23, wherein the compressed absorbent structure has a density of at least about 0.25 g/cm³ and wherein the bulk thickness of the compressed absorbent structure increases by less than about 131% after contact with 10 mls of blood over a 10 second period.

26. The method of claim 23, wherein the compressed absorbent structure has a density of at least about 0.30 g/cm³ and wherein the bulk thickness of the compressed absorbent structure increases by less than about 165% after contact with 10 mls of blood over a 10 second period.

27. The method of claim 23, wherein the compressed absorbent structure has a density of at least about 0.35 g/cm³ and wherein the bulk thickness of the compressed absorbent structure increases by less than about 211% after contact with 10 mls of blood over a 10 second period.

28. A feminine hygiene product comprising:
a compressed absorbent core comprising fluff pulp comprising cellulosic fiber that has been oxidized with a copper or iron catalyst and a peroxide in an acidic environment within a bleaching sequence;
wherein said compressed absorbent core has a bulk thickness and a density of at least about 0.15 g/cm³;
and wherein the bulk thickness grows by less than about 59% upon insult with 10 mls of blood over a 10 second period.

29. The product of claim 28, wherein said compressed absorbent core has a density of at least about 0.20 g/cm³ and wherein the bulk thickness grows by less than about 95% upon insult with 10 mls of blood over a 10 second period.

30. The product of claim 28, wherein said compressed absorbent core has a density of at least about 0.25 g/cm³ and wherein the bulk thickness grows by less than about 165% upon insult with 10 mls of blood over a 10 second period.

31. The product of claim 28, wherein said compressed absorbent core has a density of at least about 0.30 g/cm³ and wherein the bulk thickness grows by less than about 211% upon insult with 10 mls of blood over a 10 second period.

32. The product of claim 28, further comprising a topsheet and a backsheet.

33. The product of claim 32, wherein said compressed absorbent core further comprises a super absorbent polymer.

34. The product of claim 28, wherein said compressed absorbent core is at least about 5% thinner upon insult with 10 mls of blood over a 10 second period than a structure comprising fluff pulp comprising cellulosic fiber that has not been oxidized with a copper or iron catalyst and a peroxide in an acidic environment within a bleaching sequence.

35. The product of claim 28, wherein said compressed absorbent core is at least about 10% thinner upon insult with 10 mls of blood over a 10 second period than a structure comprising cellulosic fiber that has not been oxidized with a copper or iron catalyst and a peroxide in an acidic environment within a bleaching sequence.

36. The product of claim 28, wherein said compressed absorbent core is at least about 15% thinner upon insult with 10 mls of blood over a 10 second period than a structure comprising cellulosic fiber that has not been oxidized with a copper or iron catalyst and a peroxide in an acidic environment within a bleaching sequence.

37. A method of making a dimensionally stable absorbent structure for contacting blood and blood products comprising:
fluffing pulp comprising cellulosic fiber that has been oxidized with a copper or iron catalyst and a peroxide in an acidic environment within a bleaching sequence;
forming an absorbent structure including the fluffed pulp; and
compressing the absorbent structure to a density of at least about 0.25 g/cm³.

38. The method of claim 37, wherein the structure is compressed to a density of at least about 0.30 g/cm³.

39. The method of claim 37, wherein the structure is compressed to a density of at least about 0.35 g/cm³.

40. The method of claim 37, wherein the structure is compressed to a density of at least about 0.45 g/cm³.

41. The method of claim 37, wherein the structure is compressed to a density of at least about 0.5 g/cm³.

42. The method of claim 37, further comprising adding a super absorbent polymer to the fluffed pulp.

43. The method of claim 42, further comprising adding a topsheet and a backsheet to the fluffed pulp to form an absorbent product.
